(19) United States
(12) Patent Application Publication
(10) Pub. No.: US 2023/0189733 A1
(43) Pub. Date: Jun. 22, 2023

(54) METHOD FOR BREEDING NEW GERMPLASM OF CLUBROOT-RESISTANT ORANGE-HEADING CHINESE CABBAGE

(71) Applicant: NORTH WEST AGRICULTURE AND FORESTRY UNIVERSITY, Xianyang (CN)

(72) Inventors: Lugang ZHANG, Xianyang (CN); Qianqian LU, Xianyang (CN); Yan MENG, Xianyang (CN); Ninan ZHANG, Xianyang (CN); Yaxiu WANG, Xianyang (CN); Yuting HE, Xianyang (CN); Xuefen ZHANG, Xianyang (CN); Xiaoyuan LIU, Xianyang (CN)

(73) Assignee: NORTH WEST AGRICULTURE AND FORESTRY UNIVERSITY, Xianyang (CN)

(21) Appl. No.: 18/047,286

(22) Filed: Oct. 18, 2022

Related U.S. Application Data
(63) Continuation of application No. PCT/CN21/81285, filed on Mar. 17, 2021.

(30) Foreign Application Priority Data
Jan. 28, 2021 (CN) 202110119706.X

(51) Int. Cl. A01H 1/00; A01H 6/20

(52) U.S. Cl. CPC A01H 1/1255; A01H 6/204

(57) ABSTRACT

A method for breeding a new germplasm of clubroot-resistant orange-heading Chinese cabbage includes: (1) obtaining F₁ by hybridizing orange-heading Chinese cabbage as a female parent and clubroot-resistant Chinese cabbage as a male parent, obtaining F₂ by selfing F₁ individual plants, planting F₂ populations, and extracting DNA from the individual plants of F₂; (2) observing horticultural traits of F₂, and performing observation and statistics of head color phenotypic traits through cutting head; (3) performing PCR amplification with DNA of F₂ using a dominant orange gene marker Br530 and a clubroot-resistant marker SC2930-T/SC2930Q, and identifying a genotype of the individual plants; (4) performing a comprehensive evaluation according to the results of marker detection and the observation of horticultural traits, selecting double-site homozygous clubroot-resistant orange-heading Chinese cabbage plants, and selfing the individual plants for 2 consecutive generations; and (5) obtaining a new germplasm of clubroot-resistant orange-heading Chinese cabbage after selfing for 3 generations.

### CROSS REFERENCE TO THE RELATED APPLICATIONS

This application is a continuation application of International Application No. PCT/CN2021/081285, filed on Mar. 17, 2021, which is based upon and claims priority to Chinese Patent Application No. 202110119706.X, filed on Jan. 28, 2021, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBZYGJ030-Sequence-Listing.xml, created on 09/02/2022, and is 10,348 bytes in size.

### TECHNICAL FIELD

The present invention belongs to the field of vegetable variety breeding and germplasm resource innovation, and particularly relates to a method for breeding a new germplasm of clubroot-resistant orange-heading Chinese cabbage.

### BACKGROUND

Chinese cabbage (Brassica campestris syn. rapa ssp. pekinensis) is one of the important vegetable crops of Brassica genus in cruciferae family. With the improvement of living standards, people pay more attention to the quality of Chinese cabbage, that is, Chinese cabbage is required to have both high quality and resistance to diseases. However, high-quality Chinese cabbage is susceptible to diseases; the application of pesticides will not only lead to environmental pollution, but also cause vegetables to develop drug resistance in case of long-term application. Therefore, the innovation of high-quality disease-resistant Chinese cabbage germplasms has become a research hotspot in recent years.

In recent years, the orange-heading Chinese cabbage cultivated by breeders not only has bright color, but also contains higher carotenoids with soft texture, delicious flavor and rich nutritional quality. In order to accelerate the breeding of orange-heading Chinese cabbage, domestic and foreign scholars have made great efforts on molecular markers of orange-heading Chinese cabbage. QI Wang obtained a molecular marker SC82-591 with a genetic distance of 3.7 cM from an orange head gene of Chinese cabbage. Feng, et al. obtained a molecular marker syau15 with a genetic distance of 1.3 cM; Zhang et al. obtained molecular markers InDel2 and Br-InDel1 with linkage distances of 0.1 and 0.2 cM by locating a Br-or gene of orange-heading Chinese cabbage in a 16.7 kb interval of an A09 linkage group, further designed an orange head marker based on differences in a BrCRTISO promoter sequence between orange-heading Chinese cabbage and common Chinese cabbage, and proved that the marker was a co-separation marker with an orange trait. L I Peirong, et al. discovered 53 SNPs and 6 base pairs deleted in the coding region of the Br-or gene in an orange-leaf material at the cDNA level, and developed a molecular marker GAA2 based on the deletion of 6 bp for assisted selection breeding.

Clubroot is an obligate parasite devastating soil-borne disease caused by infection of plasmodiophora brassicae woron. In recent years, clubroot has attacked many places in China, and has become a main disease in the production of Chinese cabbage. The clubroot cannot be fundamentally prevented through field management and biochemical control. Breeding Chinese cabbage varieties resistant to clubroot is an important measure to solve the problem of clubroot. YANG Zheng, et al. identified 78 clubroot-resistant molecular markers in 78 Chinese cabbage materials using the CRa and CRb molecular marker primer sets of Chinese cabbage clubroot-resistant gene, and screened out 17 homozygous disease-resistant site materials. ZHU Mingzhao, et al. identified clubroot-resistant molecular markers in 24 Chinese cabbage materials and obtained 5 materials with excellent resistance to various physiological races of plasmodiophora. CHEN Lixiao, et al. took 10 clubroot-resistant Chinese cabbage varieties as test materials, and screened the DH group using the specific molecular marker CRaEXON4-3 of the clubroot-resistant gene CRa to obtain 27 plants carrying the resistance gene. Molecular marker assisted selection breeding is a plant selective breeding method that judges the existence of a target gene and select the target gene by analyzing a genotype of the molecular marker closely linked to the target gene at a molecular level.

So far, there is no related report on the application of molecular marker-assisted selection of orange leaf heads and resistance to clubroot simultaneously.

### SUMMARY

In view of the above-mentioned description that there is no report on aggregating the resistance to clubroot disease with orange-heading Chinese cabbage using molecular marker selection in the prior research, the present invention is intended to provide a method for breeding a new germplasm of clubroot-resistant orange-head Chinese cabbage; by combining molecular marker-assisted selection with field trait selection, and performing genotype screening and continuous selfing of individual plants in Chinese cabbage segregation populations, the Chinese cabbage selfing lines containing both a Chinese cabbage orange gene and a clubroot-resistant gene are obtained, thereby creating a high-quality germplasm resource for breeding Chinese cabbage.

The present invention adopts the following technical solutions:

A method for breeding a new germplasm of clubroot-resistant orange-heading Chinese cabbage includes the following steps:

(1) obtaining F₁ by hybridizing orange-heading Chinese cabbage used as a female parent and clubroot-resistant Chinese cabbage used as a male parent, obtaining F₂ by selfing F₁ individual plants, planting F₂ populations and numbering the individual plants of F₂ and extracting DNA of each plant during a seedling stage;

(2) transplanting the F₂ populations in the field, and observing horticultural traits and performing observation and statistics of head color phenotype traits through cutting head in the late heading stage of F₂;

(3) performing PCR amplification with DNA of F₂ individual plants using a dominant orange gene marker Br530 and an clubroot-resistant marker SC2930-T/SC2930Q, and identifying the genotype of the F₂ individual plants;

(4) performing comprehensive evaluation according to the results of two gene markers detected and the observation results of horticultural traits, then selecting the double-site homozygous plants at both clubroot-resistant and orange-heading Chinese cabbage to selfbreed for 2 consecutive generations;

(5) obtaining selfing lines with stable horticultural traits and the homozygous clubroot-resistant orange-heading Chinese cabbage plants by selfing the double-site homozygous clubroot-resistant orange-heading Chinese cabbage plants for 3 generations.

Further, the dominant orange gene marker Br530 is shown in SEQ ID NO: 1 as follows:

CAGAAACATCAGGGTTGAAATCTAAACCCAGAAAATAAACCCAATATGG
TATAGGTTTACCCGTGGGTACCCAAAGTATTATCTTATTTATTCTGAAG
ATCATGTAAAACTCATTTATGGTTTTAACGAGAAAACTTGTAAAGTTGT
TTTTGTGGTTTTAGCGGAAATTTTTCTTTTTGCGGTTTTTGGTCGGTAA
TTTTATTTTGTGGCTTGGTTGGAAAACTCATTTTTGCGGTTTGCGGGAA
AAATAATCTTTCTGGTTTTGACGAAAAAATTCGGTTTTACGGTTTTTGC
GAGAAAATTCGGTTTAGCAGTTTTGGCAGGAAACCTCGCTTTTGCGGTT
TTGGCGGAAAAACTCGTTTTTGATTTTGACGGAAAAACTTGTTTTTACG
GTTTTGGGGAAACTCGGTTTTCGGCTTTGACGGGAAAACTCGATTTTTC
GATTTTGGCGGGAAAACTCGATTTTGCGGTTTTGGCGGGAAAACTCGGT
TTTTCTGTTTTGGCGGAAAAACCATGTTTTTCGC TTTCGGCAGTAA.

Further, PCR-specific amplification primers for the dominant orange gene marker Br530 are as follows:

upstream primer (F):
(SEQ ID NO: 2)
5′-CAGAAACATCAGGGTTGAAATC-3′;
and
downstream primer (R):
(SEQ ID NO: 3)
5′-TTACTGCCGAAAGCGAAA-3′.

Further, PCR-specific amplification primers for clubroot-resistant marker SC2930-T are as follows:

upstream primer (F):
(SEQ ID NO: 4)
5′-TAGACCTTTTTTTTGTCTTTTTTTTTACCT-3′;
and
downstream primer (R):
(SEQ ID NO: 5)
5′-AAGGCCATAGAAATCAGGTC-3′.

Further, PCR-specific amplification primers for clubroot-resistant marker SC2930Q are as follows:

upstream primer (F):
(SEQ ID NO: 6)
5′-CAGACTAGACTTTTTGTCATTTAGACT-3′;
and
downstream primer (R):
(SEQ ID NO: 7)
5′-AAGGCCATAGAAATCAGGTC-3′.

Further, a PCR amplification system is as follows:

a total volume is 10 μL, including 5 μL of 2×Taq Master Mix for PAGE (Dining), 1 μL of DNA, 0.5 μL of upstream primer, 0.5 μL of downstream primer, and the remaining amount of ddH₂O.

Further, a PCR-specific amplification procedure for the dominant orange gene marker Br530 is as follows:

95° C. for 3 min,

95° C. for 30 s,

58° C. for 30 s,

72° C. for 1 min,

38 cycles,

72° C. for 10 min,

6° C.

∞.

Further, a PCR-specific amplification procedure for the clubroot-resistant marker SC2930-T/SC2930Q is as follows:

94° C. for 3 min,

94° C. for 1 min,

55° C. for 1.5 min,

72° C. for 2 min,

30 cycles,

72° C. for 7 min,

6° C.,

∞.

Further, orange-heading Chinese cabbage 13S93 is taken as a female parent.

Further, clubroot-resistant Chinese cabbage “Jinjin” is taken as a male parent.

In summary, owing to these technical solutions, the present invention has the the following beneficial effects:

The orange phenotype of Chinese cabbage is controlled by a single recessive gene, a ratio of orange-head individual plants to white-head individual plants in the F2 generation population is about 1:3 in the present invention, and it is fully indicated that the orange trait is independent inheritance controlled by a recessive single gene, conforming to the Mendel's laws of inheritance. Several different markers have been developed based on sequence differences between the Br-or genes of white-heading and orange-heading Chinese cabbage varieties. But these markers cannot be universally suitable for all varieties, because these sequences are not exclusively present in orange-heading Chinese cabbage, and such deletion has also been detected in other white-heading Chinese cabbage. The molecular markers used in the present invention are developed based on the insertion of a large fragment of 501 bp at the 3′ end of the Brcrtiso gene of orange-heading Chinese cabbage as compared with the Brcrtiso gene of the white-heading Chinese cabbage, which can directly detect the functional variation of the gene. The results show that 146 individual plants carrying the orange head gene were detected by the molecular markers, and 57 individual plants had not amplified orange marker. The separation of the molecular markers also conforms to the Mendel's laws of inheritance.

The disease-resistant gene contained in the Chinese cabbage materials can be rapidly detected by using the molecular marker specific to the clubroot-resistant gene. In the present invention, among the 203 plants detected using the CRa-specific molecular marker, there are 61 plants with the homozygous disease-resistant marker, 102 plants with resistant and susceptible co-dominant marker and 36 plants with homozygous susceptible marker, respectively, with a ratio close to 1:2:1. By using the method of the present invention, the clubroot-resistant homozygous germplasm resource can be obtained and further applied in the practice of clubroot-resistant breeding.

In the present invention, the genes that control the orange head are a pair of recessive genes located in the A9 linkage group, the genes CRa that control the clubroot disease are a pair of dominant genes located in the A3 linkage group. For the free combination of non-allelic genes on non-homologous chromosomes in the offspring gene recombination, a ratio of the four phenotypes (white-heading resistant:orange-heading resistant:white-heading susceptible:orange-heading susceptible) in F₂ is theoretically 9:3:3:1. In the marker identification combined with phenotype statistics, there are 104 white-heading resistant individual plants, 34 orange-heading resistant individual plants, 45 white-heading susceptible individual plants and 16 orange-heading susceptible individual plants. The chi-square test conformed to the law of free combination (x²=3.461<x² (0.05, 3)=7.815) at 9:3:3:1, firstly, 7 individual plants were screened out of 34 orange-heading resistant individual plants as homozygous clubroot-resistant orange-heading Chinese cabbage plants, then 3 stable selfing lines were obtained by selfing and observation for two consecutive generations of them, which indicates that the molecular marker-assisted selection method of the present invention is simple and efficient.

To sum up, according to the present invention, the individual plants of Chinese cabbage segregation populations are screened by combining molecular marker-assisted selection with field trait selection, the objective of selecting a Chinese cabbage selfing line containing both a Chinese cabbage orange gene and an clubroot-resistant gene is achieved, which provide a new method for creating a high-quality germplasm resource for Chinese cabbage breeding. In addition, the present invention realizes the direct selection of genotypes using molecular marker-assisted selection for polymerization breeding, which is fast and accurate, and not susceptible to the environment; the method can be performed in any period of plant growth, so the breeding duration is effectively shortened.

### BRIEF DESCRIPTION OF THE DRAWINGS

In order to explain the technical solutions of the embodiments of the present invention more clearly, the accompanying drawings to be used in describing the embodiments will be briefly introduced below. It should be understood that, the accompanying drawings below only show some embodiments of the present invention, rather than being deemed as a limitation to the scope. For those of ordinary skills in the art, other related drawings can also be obtained based on these drawings without making creative efforts.

FIG. 1 is a flow chart of breeding of the present invention.

FIG. 2 is a flow chart of breeding of an embodiment.

FIG. 3 is amplification results of a primer Br530 in 22 samples; M represents the molecular weight standard DL2000; and the number represents the serial number of individual plants.

FIG. 4 is amplification results of a primer SC2930T in 22 samples; M represents the molecular weight standard DL2000; and the number represents the serial number of individual plants.

FIG. 5 is amplification results of a primer SC2930Q in 22 samples; M represents the molecular weight standard DL2000; and the number represents the serial number of individual plants.

FIG. 6 is an image of a head of clubroot-resistant orange-heading Chinese cabbage.

FIG. 7 is an image of a cross section of a head of clubroot-resistant orange-heading Chinese cabbage.

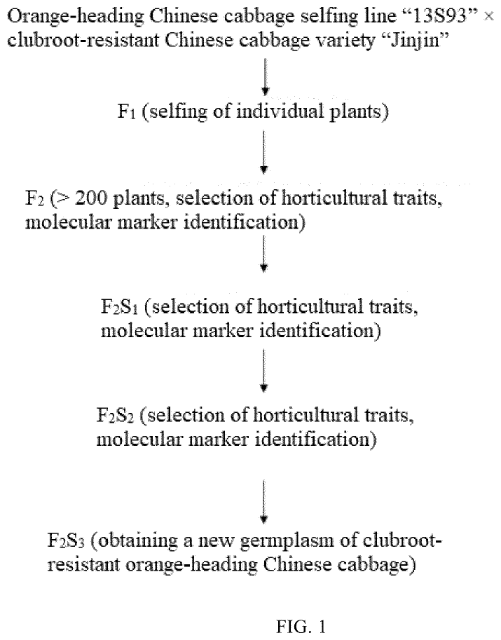

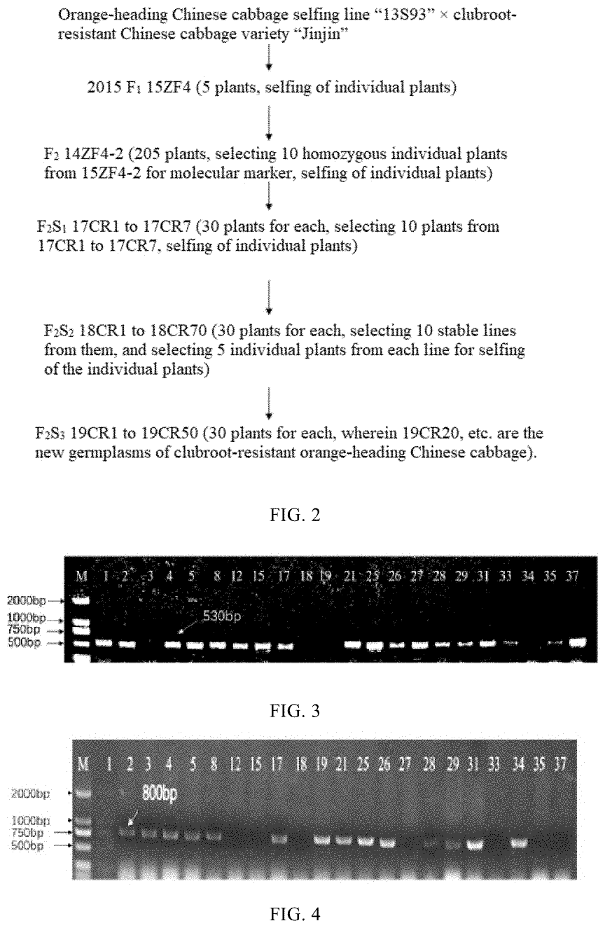

### DETAILED DESCRIPTION OF THE EMBODIMENTS

In order to make the objectives, technical solutions and advantages of the present invention more clearly, the present invention will be further explained in detail below in combination with the accompanying drawings and embodiments. It should be understood that the specific embodiments described here are only used for explaining, rather than limiting, the present invention, that is, the described embodiments are only parts of, rather than all of, the embodiments of the present invention. Usually, the components of the embodiments of the present invention described and shown in the accompanying drawings here may be arranged and designed in various configurations.

Therefore, the detailed description of the embodiments of the present invention provided in the accompanying drawings only represent the selected embodiments of the present invention, but is not intended to limit the claimed scope of the present invention. On the basis of the embodiments of the present invention, all the other embodiments obtained by those skilled in the art without making creative efforts will fall within the protection scope of the present invention.

It should be noted that the terms “first”, “second” and other relational terms are only used to distinguish one entity or operation from another, rather than necessarily requiring or implying any such actual relationship or sequence between these entities or operations. Besides, the terms “including/comprising”, “containing” or any other variants thereof are intended to encompass non-exclusive inclusion, so that a process, method, article or device including a series of elements includes not only those elements, but also other elements not expressly listed, or also includes elements inherent to such process, method, article or device. Without further limitation, an element confined by the phrase “including/comprising a/an . . . ” does not exclude the presence of additional identical elements in a process, method, article or device that includes the element.

The orange-heading Chinese cabbage selfing line 13S93 in the following example is an orange-heading Chinese cabbage selfing line bred by the research group, and the plant features are yellow-green outer leaves, semi-erect plant, half-open head, medium height, cannonball shape, and orange head leaves.

The clubroot-resistant Chinese cabbage variety “Jinjin” in the following example is a commercial variety.

It should be noted that the orange-heading Chinese cabbage selfing line 13S93 and the clubroot-resistant Chinese cabbage variety “Jinjin” (as male parent and female parent respectively) are taken as examples, but the varieties are not limited to them.

The features and performances of the present invention will be further described in details in combination with the embodiments.

### Example

A preferred embodiment of the present invention provides a method for breeding a new germplasm of clubroot-resistant orange-heading Chinese cabbage, including the following specific steps:

1. F₁ was obtained by hybridizing the orange-heading Chinese cabbage selfing line 13S93 taken as a female parent and the clubroot-resistant Chinese cabbage “Jinjin” taken as a male parent.

2. F₁ (15ZF4) individual plants were planted in the spring of the next year to obtain F₂ by selfing, and a total of 205 plants of F₂ segregation population 15ZF4-2 were planted; and the Chinese cabbage was planted in a flat field, the fertility was uniform, and the soil is alkaline soil. The Chinese cabbage seeds were sowed in the hole plate and transplanted to an open field after sowed 30 d. The plant spacing and row spacing was 45 cm and 50 cm respectively, and conventional field management was performed.

At the seedling stage of the Chinese cabbage, each individual plant was numbered by inserting a plate in the field, the DNAs of them was extracted; 750 μL of 1×CTAB extract was added to a 1.5 ml centrifuge tube, the extract composition was as follows: 2% CTAB, 100 mmol/L Tris-HCl, 20 mmol/L EDTA and 1.4 mol/L NaCl, lastly 8 μL of β-mercaptoethanol was added and shaken well; 0.2 g of fresh leaves with a main vein removed was sampled from each individual plant and then ground into powder after liquid nitrogen was added, then the extract in the 1.5 ml centrifuge tube was added to a mortar, the mixture was mixed evenly and then transferred to the centrifuge tube, and the centrifuge tube was shaken constantly; after that, the centrifuge tube was placed into a 65° C. water bath and shaken every a few minutes in the process, and the water bath lasted for 30 minutes; the centrifuge tube was taken out, an isometric phenol:chloroform:isoamylol mixture was added at a ratio of phenol:chloroform:isoamyl alcohol=25:24:1, and the centrifuge tube was shaken well for 10 min and then centrifugated at a room temperature at 12,000 r/min for 10 min; after an upper liquid phase was transferred to another centrifuge tube, an isometric chloroform:isoamylol mixture at a proportion of 24:1 was added, and the centrifuge tube was shaken well for 10 min and then centrifugated at a room temperature at 12,000 r/min for 10 min; a supernatant was collected, pre-cooled absolute ethanol was added in a volume 2 times that of the supernatant, DNA was agglomerated after mixing evenly, precipitation was performed at −20° C. for 30 min, and centrifugation was performed at 4° C. at 12,000 r/min for 10 min; after the supernatant was discarded, the precipitate was washed 2-3 times by adding 1 ml of 75% ethanol, and the precipitate was air-dried at a room temperature; the DNA was dissolved by adding 400-500 μL of TE buffer, 1.5 μL of 10 μg/L RNaseA was added until a final concentration reaches 10 μg/mL, then mixed evenly and performed at 37° C. for 30 min; after the DNA was completely dissolved, 40-50 μL of 3 mol/L NaAC solution and isometric isopropanol were added and shaken well to fully precipitate the DNA, and was performed in ice bath for 15 min; after centrifugation at 4° C. and 12,000 r/min for 10 min, the supernatant was discarded, the precipitate was washed 1-2 times by adding 1 ml of 75% ethanol, then 1,200 μL of DNA stock solution was added, and the DNA stock solution was prepared from 75% ethanol and 0.3 mol/L NaAC solution; it was confirmed by purity detection that the extracted DNA was better purified, the extracted DNA was generally diluted with 400-500 μL of sterile ddH₂O and stored at 4° C. for later use.

PCR amplification was performed using primers of a dominant orange gene marker Br530 and a clubroot-resistant marker SC2930-T/SC2930Q, and genotypes of the individual plants were identified.

The PCR-specific amplification primers for the dominant orange gene marker Br530 were as follows:

upstream primer (F):
(SEQ ID NO: 2)
5′-CAGAAACATCAGGGTTGAAATC-3′;
and
downstream primer (R):
(SEQ ID NO: 3)
5′-TTACTGCCGAAAGCGAAA-3′.

PCR-specific amplification primers for the clubroot-resistant marker SC2930-T were as follows:

upstream primer (F):
(SEQ ID NO: 4)
5′-TAGACCTTTTTTTTGTCTTTTTTTTTACCT-3′;
and
downstream primer (R):
(SEQ ID NO: 5)
5′-AAGGCCATAGAAATCAGGTC-3′.

PCR-specific amplification primers for the clubroot-resistant marker SC2930Q were as follows:

upstream primer (F):
(SEQ ID NO: 6)
5′-CAGACTAGACTTTTTGTCATTTAGACT-3′;
and
downstream primer (R):
(SEQ ID NO: 7)
5′-AAGGCCATAGAAATCAGGTC-3′.

A PCR amplification system was as follow:

a total volume was 10 μL, including 5 μL of 2×Taq Master Mix for PAGE (Dining), 1 μL of DNA, 0.5 μL of upstream primer, 0.5 μL of downstream primer, and the remaining amount of ddH₂O.

A PCR-specific amplification procedure for the dominant orange gene marker Br530 was as follows:

95° C. for 3 min,

95° C. for 30 s,

58° C. for 30 s,

72° C. for 1 min,

38 cycles,

72° C. for 10 min,

6° C.,

∞.

A PCR-specific amplification procedure for the clubroot-resistant marker SC2930-T/SC2930Q was as follows:

94° C. for 3 min,

94° C. for 1 min,

55° C. for 1.5 min,

72° C. for 2 min,

30 cycles,

72° C. for 7 min,

6° C.,

∞.

Individual plant identification was performed on the population using the single dominant orange head marker Br530, and the results showed (FIG. 3) that there were 146 individual plants carrying the orange marker and 57 individual plants not containing orange marker. According to the chi-square test, the orange gene conformed to the Mendel's laws of inheritance at 3:1 (x²=1.026<x² (0.05, 1)=3.84). The statistics of the head color of individual plants was performed by cutting the heads in the late heading stage of Chinese cabbage, and in the phenotypic statistics, there were 51 orange heads and 152 white heads, which conform to a separation proportion of 1:3. It was indicated that orange trait was recessive inheritance to white trait. In the screening of phenotypes by combining molecular marker with head color, 51 of 203 valid individual plants were allelic homozygous orange individual plants.

It can be known from FIG. 4 and FIG. 5 that 4 plants had no amplified bands with both primers; the results of the resistant gene identification on the population using the CRa clubroot-resistant gene primers showed that 163 individual plants contained the SC2930-Q susceptible marker, where 61 individual plants only contained the SC2930Q marker, and 102 individual plants were heterozygous disease-resistant individual plants; and 138 individual plants contained the SC2930-T disease-resistant marker, where 36 individual plants only contained the SC2930-T marker, this indicated that these individual plants contained the allelic homozygous clubroot-resistant gene CRa. According to the chi-square test, the clubroot-resistant gene conformed to the Mendel's laws of inheritance of 3:1 (x²=3.392 (x² (0.05, 1)=3.84).

To sum up, in the F₂ segregation population, 51 individual plants contained the homozygous orange head gene; 102 individual plants contained the clubroot-resistant gene, and 36 individual plants contained the homozygous clubroot-resistant gene, wherein to, 34 individual plants were clubroot-resistant orange-heading Chinese cabbage, with the individual plant numbers as 4, 5, 8, 17, 21, 39, 45, 50, 51, 54, 59, 62, 75, 76, 83, 105, 107, 119, 124, 125, 128, 134, 143, 147, 154, 155, 156, 161, 165, 168, 169, 197, 200 and 202, where 7 individual plants were homozygous clubroot-resistant orange-heading Chinese cabbage, with the individual plant numbers as 8, 17, 50, 107, 134, 155 and 168, and all the 7 individual plants were reserved for selfing in next year.

3. In the spring of the next year, the 7 individual plants selected and reserved in the previous year were cultivated, and 7 pieces of the seeds of F₂S₁ population were obtained by the individual plants selfing and numbered as 17CR1 to 17CR7, respectively. The seeds of the F₂S₁ population (30 plants for each of 17CR1 to 17CR7, and a total of 210 plants) were sowed and planted in the field in the autumn, and their consistency of horticultural traits was observed, lastly, 10 individual plants with good heading and consistent horticultural traits were selected from each of 17CR1 to 17CR7, and they were naturally subjected to vernalization for selfing in next year.

4. In the spring of the next year, the individual plants selected from 17CR1 to 17CR7 were cultivated, and 70 pieces of the seeds of an F₂S₂ population were obtained by the individual plants (70 plants) selfing and numbered as 18CR1 to 18CR70, respectively. The seeds of the F₂S₂ population (30 plants for each of 18CR1 to 18CR70, and a total of 2,100 plants) were sowed and planted in the field in the autumn, the consistency of horticultural traits was observed, lastly, 10 stable lines were selected, and 5 individual plants selected from each line were naturally subjected to vernalization for selfing in next year.

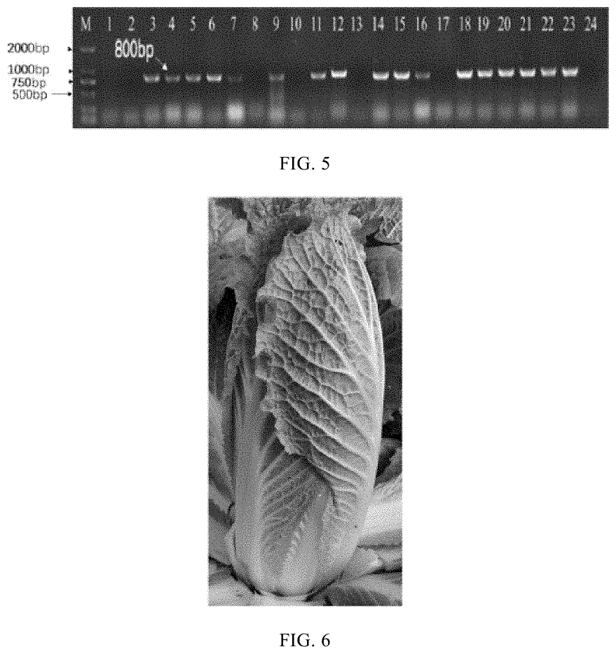

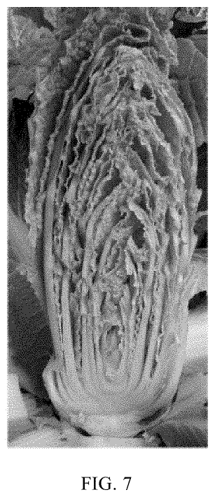

5. In the spring of the next year, 10 stable lines selected from 18CR1 to 18CR70 were cultivated, with 5 individual plants for each line, and 50 pieces of seeds of an F₂S3 population were obtained by the individual plants selfing and numbered as 19CR1 to 19CR50, respectively. The seeds of the F₂S₃ population (30 plants for each of 19CR1 to 19CR50, and a total of 1,500 plants) were planted in the field in the autumn, the consistency of horticultural traits was observed, it was found that 3 lines (19CR5, 19CR26 and 19CR38) had regular and consistent horticultural traits and carried the homozygous orange gene and clubroot-resistant gene, the phenotype of the new germplasm of 19CR26 clubroot-resistant orange-heading Chinese cabbage was as shown in FIG. 6 and FIG. 7.

The new germplasms 19CR26, etc. with the clubroot-resistant orange-heading Chinese cabbage bred in the present invention have regular horticultural traits and good heading, complete the aggregation of the two traits of orange trait and resistance to clubroot, and enrich the germplasm resource of Chinese cabbage, thereby laying a foundation for breeding new varieties of clubroot-resistant orange-heading Chinese cabbage.

Those described above are only the preferred embodiments of the present invention, rather than limiting the present invention; and any modification, equivalent alternation or improvement within the spirit and principle of the present invention will fall within the protection scope of the present invention.

1. A method for breeding a new germplasm of clubroot-resistant orange-heading Chinese cabbage, comprising the following steps:
(1) obtaining F₁ by hybridizing orange-heading Chinese cabbage used as a female parent and clubroot-resistant Chinese cabbage used as a male parent, obtaining F₂ by selfing F₁ individual plants, planting F₂ populations, and numbering individual plants of the F₂ populations and extracting DNA of each plant during a seedling stage;
(2) transplanting the F₂ populations in a field, and observing horticultural traits and performing observation and statistics of head color phenotype traits through cutting head in a late heading stage of the F₂ populations;
(3) performing a PCR amplification with DNA of F₂ individual plants using a dominant orange gene marker Br530 and an clubroot-resistant marker SC2930-T/SC2930Q, and identifying a genotype of the F₂ individual plants;
(4) performing a comprehensive evaluation according to results of the dominant orange gene marker Br530 and the clubroot-resistant marker SC2930-T/SC2930Q detected and observation results of the horticultural traits, then selecting double-site homozygous plants at both the clubroot-resistant Chinese cabbage and the orange-heading Chinese cabbage to selfbreed for 2 consecutive generations; and
(5) obtaining selfing lines with stable horticultural traits and homozygous clubroot-resistant orange-heading Chinese cabbage plants by selfing double-site homozygous clubroot-resistant orange-heading Chinese cabbage plants for 3 generations.

2. The method for breeding the new germplasm of the clubroot-resistant orange-heading Chinese cabbage of claim 1, wherein the dominant orange gene marker Br530 is shown in SEQ ID NO: 1 as follows:
CAGAAACATCAGGGTTGAAATCTAAACCCAGAAAATAAACCCAATATGG
TATAGGTTTACCCGTGGGTACCCAAAGTATTATCTTATTTATTCTGAAG
ATCATGTAAAACTCATTTATGGTTTTAACGAGAAAACTTGTAAAGTTGT
TTTTGTGGTTTTAGCGGAAATTTTTCTTTTTGCGGTTTTTGGTCGGTAA
TTTTATTTTGTGGCTTGGTTGGAAAACTCATTTTTGCGGTTTGCGGGAA
AAATAATCTTTCTGGTTTTGACGAAAAAATTCGGTTTTACGGTTTTTGC
GAGAAAATTCGGTTTAGCAGTTTTGGCAGGAAACCTCGCTTTTGCGGTT
TTGGCGGAAAAACTCGTTTTTGATTTTGACGGAAAAACTTGTTTTTACG
GTTTTGGGGAAACTCGGTTTTCGGCTTTGACGGGAAAACTCGATTTTTC
GATTTTGGCGGGAAAACTCGATTTTGCGGTTTTGGCGGGAAAACTCGGT
TTTTCTGTTTTGGCGGAAAAACCATGTTTTTCGCTTTC GGCAGTAA.

3. The method for breeding the new germplasm of the clubroot-resistant orange-heading Chinese cabbage of claim 1, wherein PCR-specific amplification primers for the dominant orange gene marker Br530 are as follows:
upstream primer (F):
(SEQ ID NO: 2)
5′-CAGAAACATCAGGGTTGAAATC-3′;
and
downstream primer (R):
(SEQ ID NO: 3)
5′-TTACTGCCGAAAGCGAAA-3′.

4. The method for breeding the new germplasm of the clubroot-resistant orange-heading Chinese cabbage of claim 1, wherein PCR-specific amplification primers for the clubroot-resistant marker SC2930-T are as follows:
upstream primer (F):
(SEQ ID NO: 4)
5′-TAGACCTTTTTTTTGTCTTTTTTTTTACCT-3′;
and
downstream primer (R):
(SEQ ID NO: 5)
5′-AAGGCCATAGAAATCAGGTC-3′.

5. The method for breeding the new germplasm of the clubroot-resistant orange-heading Chinese cabbage of claim 1, wherein PCR-specific amplification primers for the clubroot-resistant marker SC2930Q are as follows:
upstream primer (F):
(SEQ ID NO: 6)
5′-CAGACTAGACTTTTTGTCATTTAGACT-3′;
and
downstream primer (R):
(SEQ ID NO: 7)
5′-AAGGCCATAGAAATCAGGTC-3′.

6. The method for breeding the new germplasm of the clubroot-resistant orange-heading Chinese cabbage of claim 1, wherein a PCR amplification system is as follows:
a total volume is 10 μL, comprising 5 μL of 2×Taq Master Mix for PAGE (Dining), 1 μL of DNA, 0.5 μL of an upstream primer, 0.5 μL of a downstream primer, and a remaining amount of ddH₂O.

7. The method for breeding the new germplasm of the clubroot-resistant orange-heading Chinese cabbage of claim 1, wherein a PCR-specific amplification procedure for the dominant orange gene marker Br530 is as follows:
95° C. for 3 min,
95° C. for 30 s
58° C. for 30 s,
72° C. for 1 min,
38 cycles,
72° C. for 10 min,
6° C.,
∞.

8. The method for breeding the new germplasm of the clubroot-resistant orange-heading Chinese cabbage of claim 1, wherein a PCR-specific amplification procedure for the clubroot-resistant marker SC2930-T/SC2930Q is as follows:
94° C. for 3 min,
94° C. for 1 min,
55° C. for 1.5 min
72° C. for 2 min,
30 cycles,
72° C. for 7 min,
6° C.,
∞.


SEQUENCE LISTING (7 sequences)

SEQ ID NO: 1 - DNA, 536 bp, synthetic construct
  misc_feature 1..536: note = The sequence is synthetized.
  source 1..536: mol_type = other DNA
cagaaacatcagggttgaaatctaaacccagaaaataaacccaatatggtataggtttacccgtgggtacccaaagtattatcttatttattctgaagatcatgtaaaactcatttatggttttaacgagaaaacttgtaaagttgtttttgtggttttagcggaaatttttctttttgcggtttttggtcggtaattttattttgtggcttggttggaaaactcatttttgcggtttgcgggaaaaataatctttctggttttgacgaaaaaattcggttttacggtttttgcgagaaaattcggtttagcagttttggcaggaaacctcgcttttgcggttttggcggaaaaactcgtttttgattttgacggaaaaacttgtttttacggttttggggaaactcggttttcggctttgacgggaaaactcgatttttcgattttggcgggaaaactcgattttgcggttttggcgggaaaactcggtttttctgttttggcggaaaaaccatgtttttcgctttcggcagtaa

SEQ ID NO: 2 - DNA, 22 bp, synthetic construct
  misc_feature 1..22: note = The sequence is synthetized.
  source 1..22: mol_type = other DNA
cagaaacatcagggttgaaatc

SEQ ID NO: 3 - DNA, 18 bp, synthetic construct
  misc_feature 1..18: note = The sequence is synthetized.
  source 1..18: mol_type = other DNA
ttactgccgaaagcgaaa

SEQ ID NO: 4 - DNA, 30 bp, synthetic construct
  misc_feature 1..30: note = The sequence is synthetized.
  source 1..30: mol_type = other DNA
tagaccttttttttgtctttttttttacct

SEQ ID NO: 5 - DNA, 20 bp, synthetic construct
  misc_feature 1..20: note = The sequence is synthetized.
  source 1..20: mol_type = other DNA
aaggccatagaaatcaggtc

SEQ ID NO: 6 - DNA, 27 bp, synthetic construct
  misc_feature 1..27: note = The sequence is synthetized.
  source 1..27: mol_type = other DNA
cagactagactttttgtcatttagact

SEQ ID NO: 7 - DNA, 20 bp, synthetic construct
  misc_feature 1..20: note = The sequence is synthetized.
  source 1..20: mol_type = other DNA
aaggccatagaaatcaggtc